(19) United States
(12) Patent Application Publication
(10) Pub. No.: US 2011/0071590 A1
(43) Pub. Date: Mar. 24, 2011

(54) SACRAL NEUROSTIMULATION TO INDUCE MICTURITION IN PARAPLEGICS

(75) Inventors: Fayçal Mounaim, Montreal (CA); Mohamad Sawan, Ville Saint-Laurent (CA)

(73) Assignee: LA CORPORATION DE L'ECOLE POLYTECHNIQUE DE MONTREAL, Montreal (CA)

(21) Appl. No.: 12/886,552

(22) Filed: Sep. 20, 2010

Related U.S. Application Data
(60) Provisional application No. 61/243,640, filed on Sep. 18, 2009.

(51) Int. Cl. A61N 1/36

(52) U.S. Cl. 607/41

(57) ABSTRACT

It is an object of the present invention to provide a method and apparatus for enhanced bladder voidance which would have the advantages of a rhizotomy yet be temporary and reversible by combining low and high-frequency sacral nerve stimulation. Applicants propose a new sacral neurostimulation strategy based on a combination of nerve conduction blockade using high frequency signals and nerve stimulation using low-frequency signals. The method and apparatus enhances micturition in paraplegics by sacral neurostimulation involving a combination of a low-frequency electrical stimulation applied to one or more sacral nerves to induce bladder contraction and a high frequency electrical stimulation applied to at least one sacral nerve to cause nerve conduction blockade that prevents urethral sphincter dyssynergic contraction.

This application claims priority to U.S. application No. 61243640 filed Sep. 18, 2009.

### TECHNICAL FIELD

The present invention relates to treating urinary dysfunctions and in particular to methods and apparatuses for sacral neurostimulation that enhance micturition in patients with spinal cord injuries.

### BACKGROUND

The efficiency of bladder emptying by means of sacral neurostimulation depends on the capability to contract the bladder muscle without generating a dyssynergic contraction of the urethral sphincter (FIG. 1). In order to improve the neurostimulation selectivity, several techniques have been proposed, among which rhizotomy, anodal block and high-frequency blockade. Dorsal sacral roots rhizotomy consists of severing afferent sacral nerve roots that are involved in pathological reflex arc in suprasacral spinal cord lesions. When combined with sacral ventral root stimulation, dorsal rhizotomy abolishes detrusor overactivity. As a beneficial result, the bladder capacity and compliance are increased, the incontinence reflex is reduced, and the upper urinary tract is protected from ureteral reflux and hydronephrosis. In addition, stimulation with rhizotomy reduces the external sphincter dyssynergia, improves urine flow, and prevents autonomic dysreflexia.

In case of a complete spinal cord injury (SCI), rhizotomy is combined with an implantable sacral ventral root stimulator such as the Finetech-Brindley (also known as the VOCARE in North America) Bladder System (FIG. 2). Actually, this neurostimulation system is the only commercialised FDA-approved solution aiming for micturition in SCI patients.

In 1984, Solomonow (Solomonow M. (1984) External Control of the Neuromuscular System. IEEE Trans Biomed Eng 31 (12):752-763) demonstrated that the high frequency blockade can be achieved with an alternating stimulation and found that 600 Hz was an optimum frequency in terms of required stimulation intensity. In 1998, Shaker et al. (H. S. Shaker, L. M. Tu, S. Robin, K. Arabi, M. Hassouna, M. Sawan, M. M. Elhilali (1998) Reduction of bladder outlet resistance by selective sacral root stimulation using high-frequency blockade in dogs: an acute study. J Urol 160 (3):901-907) studied the efficiency of this technique in acute dog experiments using a neurostimulator designed by Applicants Polystim laboratory. The stimulator generated a rectangular waveform combining two frequencies (e.g. 600 Hz and 30 Hz) such that the higher frequency blocks the urethral sphincter activity and reduces dyssynergia during micturition. It is important to point out in this case, that stimulation and blockade are both applied simultaneously at the same nerve site, with the same electrode. Here, the used term “blockade” refers more to the stimulation result which is the inhibition of the sphincter muscle contraction.

According to Kilgore et al., (K. L. Kilgore, N. Bhadra (2004) Nerve conduction block utilising high-frequency alternating current. Med Biol Eng Comput 42 (3):394-406) a reversible nerve blockade can be achieved with different waveforms with frequencies from 2 to 20 kHz. It is shown that this blockade is not indirectly induced by fatigue. In addition, some simulations corroborate the hypothesis that high-frequency stimulation maintains the nerve membrane in a depolarization status. It is also stated that a frequency of 600 Hz can also achieve a complete blockade but would require high stimulation currents (higher than 4 mA). Thus, blockade with 600 Hz is probably due to a muscle fatigue mechanism rather than nerve conduction blockade. In the case of sinusoidal waveforms, low frequencies (500 Hz-1 kHz) generates CAPs at the same or sub-multiple rate. The generated CAP amplitude vanishes for frequencies beyond 1 kHz. Blockade of each axon within the nerve is also influenced by the stimulation amplitude as well as the axon diameter. Thus a graded blockade can be achieved. If applied in combination with low-frequency stimulation (at different nerve sites), selectivity with respect to axon diameter can be achieved by adjusting stimulation amplitudes. In the urinary system case, Sievert et al. (K. D. Sievert, C. A. Gleason, K. P. Jünemann, P. Alken, E. A. Tanagho (2002) Physiologic bladder evacuation with selective sacral root stimulation: sinusoidal signal and organ-specific frequency. Neurourol Urodyn 21 (1):80-91) present several animal experimental results especially the bladder and sphincter pressure responses to a sinusoidal stimulation up to 10 kHz. It is shown that sphincteric pressure is maximal around 100 Hz, and decreases drastically as the frequency is increased. Beyond 1 kHz, the sphincteric pressure becomes very low. The blockade discussed so far concerned motor action potentials induced by neurostimulation. A complete blockade of sensory activity would probably require higher stimulation amplitudes. Bhadra et al. (N. Bhadra, K. Kilgore, K. J. Gustafson (2006) High frequency electrical conduction block of the pudendal nerve. J Neural Eng 3 (2) 180-187) applied the high frequency block to the pudendal nerves of male cats and successfully blocked the EUS contractions. It is demonstrated that a complete reversible conduction block could be achieved over all tested frequencies (1 to 30 kHz) at varying stimulus amplitudes (1 to 10 V). Finally, Boger et al. (A. Boger, N. Bhadra, K. J. Gustafson (2008) Bladder voiding by combined high frequency electrical pudendal nerve block and sacral root stimulation. Neurourol Urodyn 27 (5):435-439) demonstrated effective micturition in male cats by combining sacral root stimulation with bilateral high frequency pudendal nerve block

Unfortunately, rhizotomy which is obviously irreversible, has a fundamental disadvantage which is the abolition of sexual and defecation reflexes, as well as sacral sensations if still presents in case of incomplete SCI.

### SUMMARY

It is an object of the present invention to provide a method and apparatus for enhanced bladder voidance which would have the advantages of a rhizotomy yet be temporary and reversible by combining low and high-frequency sacral nerve stimulation. Applicants propose a new sacral neurostimulation strategy based on a combination of nerve conduction blockade using high frequency signals and nerve stimulation using low-frequency signals. Essentially, low frequency signals favor contraction of the detrusor muscle of the bladder whereas high frequency signals have a dual effect. On one hand, high-frequency signals prevent passage of afferent electrical impulses that are part of a feedback loop which serves to contract the urethra during bladder contraction, thus normally preventing incontinence and on the other hand they partially inhibit efferent signals on the large fibers that arise from the low-frequency stimulation which serve to contract the urethral sphincter. The methods presented herein are an advantageous temporary and reversible alternative to rhizotomy, which implies permanent and unfortunate act of nerve dissection.

It is an object of the present invention to provide a method to enhance micturition in paraplegics by sacral neurostimulation involving a combination of a low-frequency electrical stimulation applied to one or more sacral nerves to induce bladder contraction and a high frequency electrical stimulation applied to at least one sacral nerve to cause nerve conduction blockade that prevents urethral sphincter dyssynergic contraction.

It is yet another object of the present invention to provide a neurostimulator for enhancing bladder function having at least two electrical signal sources and electrodes for coupling to sacral nerves, the neurostimulator being configured to deliver a combination of a low-frequency electrical stimulation to one or more sacral nerves to induce bladder contraction and a high frequency electrical stimulation to at least one sacral nerve to cause nerve conduction blockade that prevents urethral sphincter dyssynergic contraction.

It is still another object of the present invention to provide a neurostimulator cuff for securing around at least two nerves comprising an electrically non-conductive main body having a central portion for receiving electrical input, the main body having at least two projections defining at least two nerve channels for receiving a nerve, and wherein the nerve channels can be in an open or closed position; an electrically conductive material comprising one or more electrode engaging the central portion of the main body and distributed independently along each nerve channel to make electrical contact along the at least two nerves.

As an alternative to rhizotomy, high-frequency stimulation can be used to achieve a complete or graded block of the compound action potential (CAP) propagating on different fibres within a nerve bundle. The mechanism by which this inhibition is obtained is not well understood and three explanations are possible: high-frequency stimulation may stop the propagation of nerve action potentials, may maintain the motor end-plate (neuromuscular junction) in a refractory status, or may fatigue the target muscle.

The use of a high-frequency alternating stimulation waveform (e.g. sinusoidal) with optimum parameters, allows a blockade of the nerve activity (motor and/or sensory), that may be complete (all axons) or partial (large diameter axons only). If the blockade is complete, the effect would be equivalent to that of rhizotomy while being controlled and totally reversible. If the blockade is partial, a selective stimulation can be achieved by blocking large axons. Consequently, a stimulation strategy based on this technique would allow better micturition by increasing bladder contraction selectivity and decreasing sphincter dyssynergia without any rhizotomy. To Applicants knowledge, such a strategy has not been tested yet in the particular case of the urinary system.

Sacral root stimulation is one of the most promising techniques for bladder rehabilitation in spinal cord-injured (SCI) patients. The only commercialized implantable neurostimulator aiming for improved micturition and having obtained satisfactory results requires rhizotomy to reduce detrusor-sphincter dyssynergia (DSD). However, rhizotomy is irreversible and may abolish sexual and defecation reflexes as well as sacral sensations, if still present in case of incomplete SCI. In order to avoid rhizotomy, applicants propose a new multisite stimulation strategy applied to extradural combined (dorsal plus ventral) sacral roots, and based on nerve conduction blockade using high-frequency stimulation as an alternative to rhizotomy. This approach would allow a better micturition by increasing bladder contraction selectively and decreasing DSD. Eight (8) acute dog experiments were carried out to verify the bladder and the external urethral sphincter (EUS) responses to the proposed stimulation strategy. High-frequency blockade (1 kHz) combined with low-frequency stimulation (30 Hz) increased the average intravesical-intraurethral pressure difference up to 53 cmH2O and reduced the average intraurethral pressure with respect to baseline by up to 86%. A custom bi-cuff-electrode design has been developed to be applied around two nerves in distinct cuffs. This could provide a more efficient micturition technique for SCI patients.

### BRIEF DESCRIPTION OF THE DRAWINGS

The invention will be better understood by way of the following detailed description of embodiments of the invention with reference to the appended drawings, in which:

FIG. 1 is a schematic representation of the urinary bladder.

FIG. 2 is a schematic representation of the state of the art for bladder neurostimulation coupled to dorsal rhizotomy.

FIG. 3 illustrates possible sacral neurostimulation sites.

FIG. 4 shows a block diagram of the electrical stimulation setup used in acute animal experiments.

FIG. 5 shows various views of the bi-cuff electrode. FIG. 5A shows a plan side elevation view, FIG. 5b shows a side view in the closed position and FIG. 5C show a side view in the open position

FIG. 6 shows the external uretheral sphincter (EUS) electromyography (EMG) activity at different blockade intensities.

FIG. 7 shows one set of experimental results from neurostimulation strategies in spinalized dog #4.

FIG. 8 shows one set of experimental results from neurostimulation strategies in spinalized dog #2.

FIG. 9 shows one set of experimental results from neurostimulation strategies in spinalized dog #1.

FIG. 10 shows one set of experimental results from neurostimulation strategies in spinalized dog #5.

FIG. 11 shows a summary table of electrical inputs and pressure changes for all experiments.

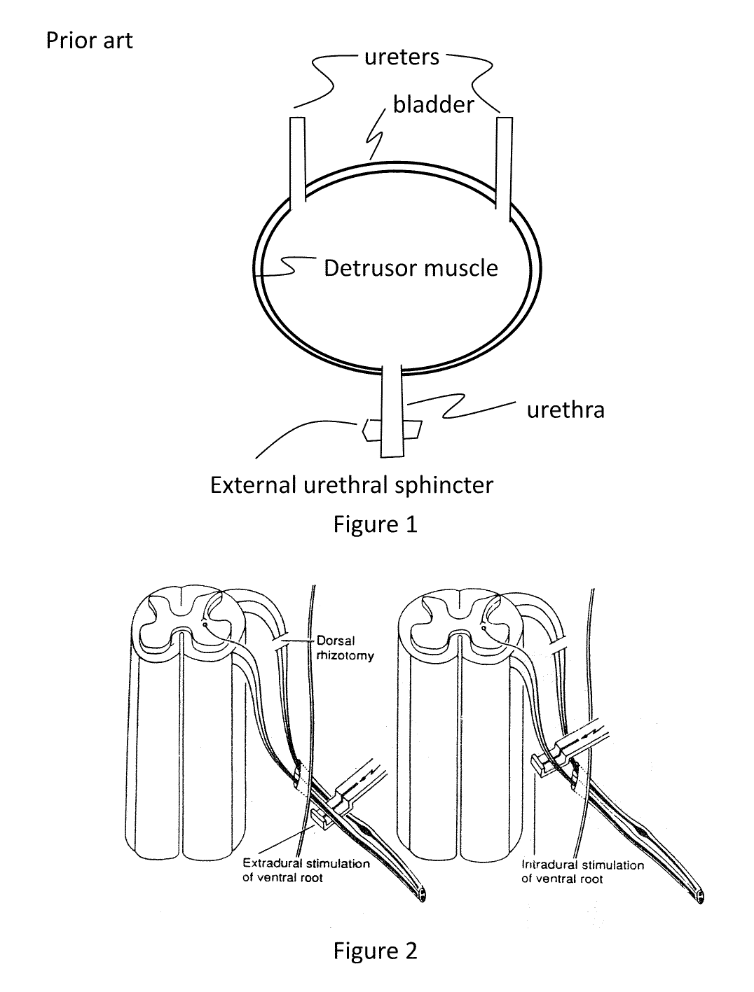

### DETAILED DESCRIPTION

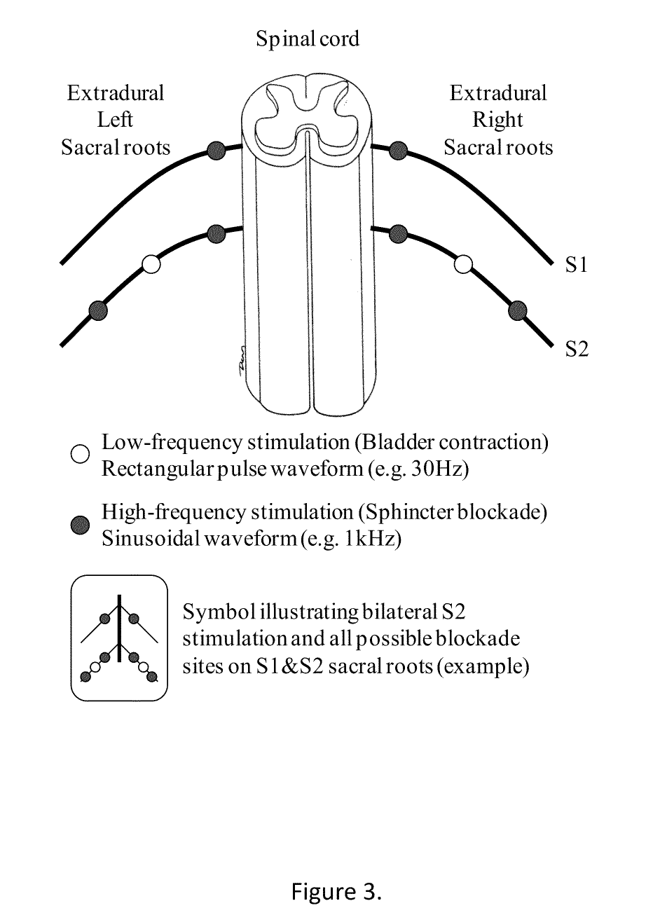

Applicants propose a new neurostimulation strategy based on the hypothesis stated previously. A low-frequency (e.g. 30 Hz) monophasic current stimulation is applied, unilaterally or bilaterally, to S2 sacral nerve(s) (or S1 eventually) in order to induce a satisfactory contraction of the bladder muscle. The degree of contraction can be modulated by adjusting the amplitude and pulse width of stimulation. In most cases, detrusor contractions are present and the EUS contracts as well. The stimulation-evoked EUS contraction can be triggered by direct and/or reflex mechanisms due to efferent and/or afferent fibres activation respectively. Both types of EUS activation can be avoided by blocking axons innervating the EUS with high-frequency stimulation. In the example of FIG. 3, a sinusoidal waveform at 1 kHz is chosen. In order to eliminate direct EUS activation, a selective blockade can be applied distally (between the low-frequency stimulation site and the EUS), whereas for reflex EUS activation, a complete blockade can be applied proximally (between the low-frequency stimulation site and the spinal cord). However, reflex EUS activation may involve sacral root(s) other than the one(s) stimulated by the low-frequency, which should be blocked as well in this case. It should be noted that blocking at all sites as shown in FIG. 3 is just for illustration purposes. Anatomically, the lower urinary tract innervations are the same from one animal to another but there is a functional variability. It is possible that one type of the direct or reflex EUS activation mechanisms is dominant, or that only one blockade site is sufficient. Conventional sacral nerve stimulation in patients with incomplete SCI may lead to pain perception. Rhizotomy can be a way to avoid the stimulation-evoked pain but will probably not be considered if important reflexes and sensations are still present. With the proposed stimulation strategy, proximal high frequency stimulation can achieve a complete blockade of sensory activity and consequently prevent pain sensation as well.

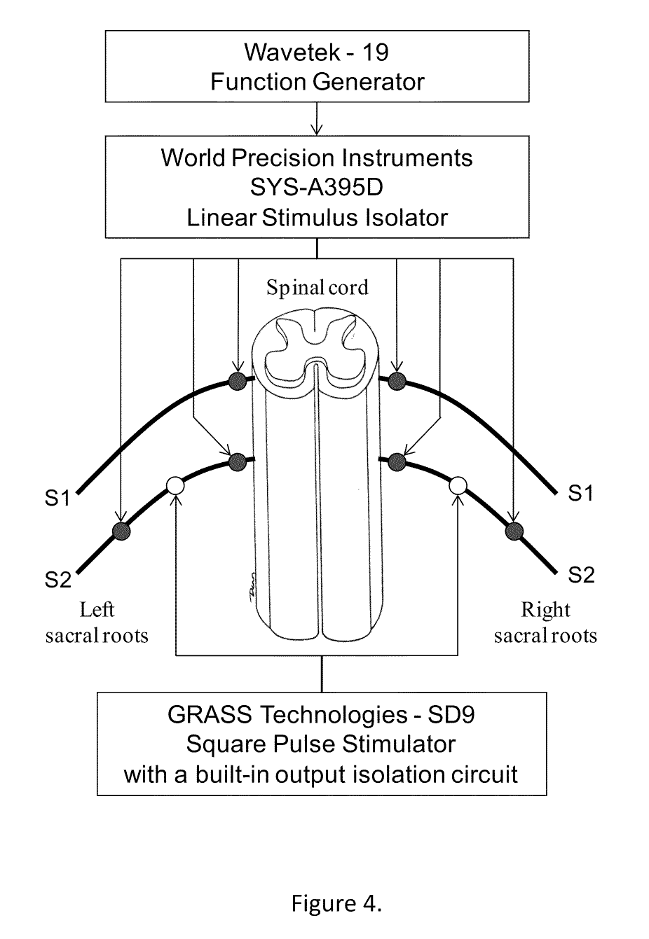

FIG. 4 shows a square pulse stimulator (GRASS Technologies—SD9) with built-in output isolation circuit was used to apply a monophasic low-frequency voltage stimulation to sacral nerve S2 (or possibly S1), unilaterally or bilaterally. The pulse frequency was fixed to 30 Hz, the pulse width was 300 μs in most experiments, while the pulse amplitude was adjusted to the minimum value that produced a satisfactory intravesical pressure (about 40 cmH2O). Tripolar electrode configuration was preferred in general.

A function generator (Wavetek—19) was used to produce the high-frequency AC voltage waveform that is converted into current with a linear stimulus isolator (World Precision Instruments—SYS-A395D). The resulting current was delivered to one or multiple blockade sites on sacral nerves S1 and/or S2 using bipolar electrode configuration (FIG. 4). The stimulation amplitude was adjusted to the minimum value that produced a satisfactory blockade of the EUS. Frequency of 1 kHz has been chosen because with increasing frequency, the voltage required to achieve a complete block increases, and so does the stimulation current for a given electrode impedance. Consequently, such combination of high frequency and high stimulation current leads to high current consumption and high supply voltage requirements that are difficult to meet for a chronic implantable neurostimulation device. However, it has previously been shown that the charge per phase required for a complete block decreases with increasing frequency. If applicants keep the same amount of charge injection per phase (CIP) using a sinusoidal waveform, stimulation at 2 kHz would require twice the current amplitude of that at 1 kHz. By extrapolation, the mean required CIP would be about 0.65 and 0.4 μC for 1 and 2 kHz respectively. So the required blockade current amplitude at 2 kHz would be about (2×0.4)/(1×0.65)=1.23 times that of 1 kHz. Thus using higher frequency for blockade is not necessarily advantageous with respect to stimulation intensity. In all experimental stimulations using both high and low-frequency signals, current densities ranged from 5 μA to 5 mA. In the case of low-frequency stimulation, a Grass Technologies SD9 Square Pulse Stimulator was used to generate electrical signals. In the case of high-frequency stimulations, a World Precision Instruments Linear Stimulus Isolator (Sys-A395D) was used.

### Cuff-Electrodes

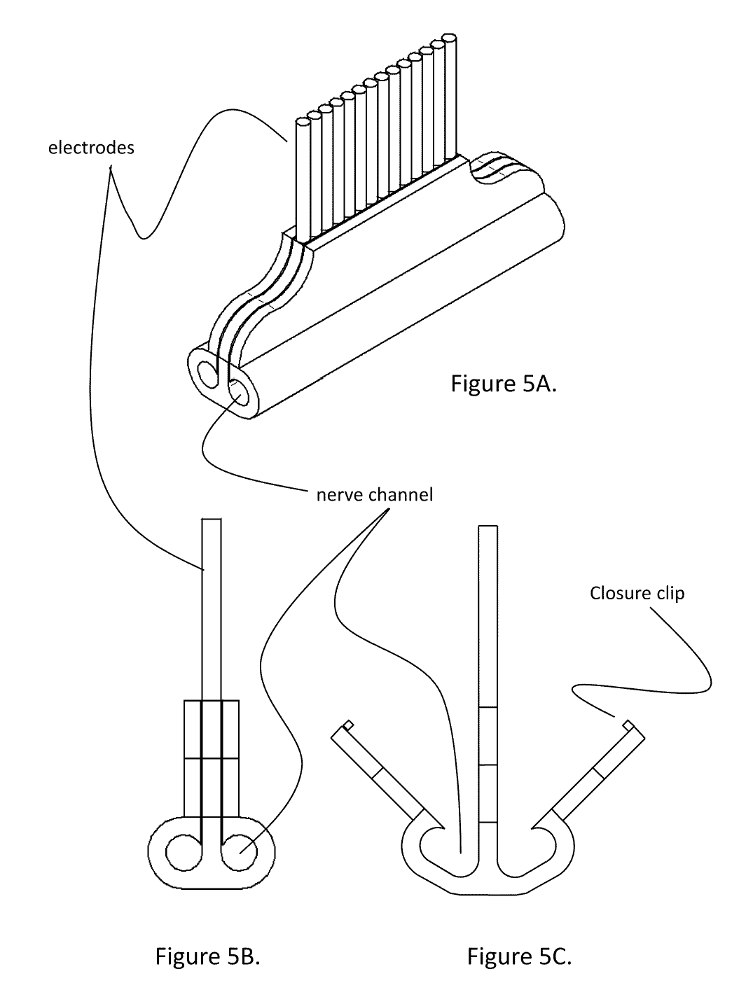

FIG. 5 shows various views of the bi-cuff electrode. FIG. 5A shows a side elevation view. FIG. 5b shows a side view in the closed position and FIG. 5C The electrode is made of hydrophilic vinyl polysiloxane material. Stainless steel wires are embedded inside the cuff wall but exposed at the inner surface of the cuff. Exposed wires serve as electrode contacts. The cuff edges are attached together with a small staple. The resulting electrode can host two nerves in distinct cuffs.

In previous chronic studies, applicants used split-cylinder cuff electrodes with a shape memory alloy (SMA) armature embedded inside the cuff wall. The electrode cuff is moulded in a biocompatible silicone, and the electrode contacts are platinum foil bands welded to leads made of multi-strands stainless steel wires coated with Teflon. The SMA electrode is easy to manipulate at low temperature, but it automatically recovers its original shape (cylindrical around the nerve) when heated at body temperature. However, despite the advantage of such an innovative design, the production of the electrodes remains laborious and costly, especially that Applicants strategy requires multiple electrodes.

For Applicants acute experiments, we proposed a simpler electrode design, yet very practical and efficient. Instead of silicone, applicants used a hydrophilic vinyl polysiloxane material used for dental impressions (DENTSPLY/CAULK, Reprosil® Light Orange), that is much easier and faster to prepare. The stainless steel wires are embedded inside the cuff wall but exposed at the inner surface of the cuff by removing the Teflon coat. Thus the exposed wires serve as electrode contacts and no platinum is used. Without any SMA armature, the resulting Reprosile cuff-electrode already offers interesting mechanical properties. It is easy to manipulate and it also recovers its original shape to a certain degree, at least for the time of the acute experiment. Keeping the cuff opened by pulling apart its two edges, the nerve can be easily inserted inside. As soon as the cuff is released, it self-closes around the nerve. In order to maintain the installed electrode closed and stable for the duration of the experiment, the cuff edges are attached together with a closure clip which can consist of a small staple or sutures.

In addition, instead of using a dedicated cuff for each stimulation or blockade site, applicants proposed to merge the cuffs that are placed on the same nerve. In other words, only one cuff is placed around each sacral nerve. If on a single nerve for example, one tripolar stimulation site and two bipolar (distal and proximal) blockade sites are required, then the cuff-electrode must offer 7 different contacts. With a contact width of less than 1 mm, and an inter-electrode distance of 1 mm, the maximum cuff length is 15 mm. The cuff inner diameter must be slightly larger than the nerve diameter (between 1 and 2 mm). Moreover, given that Applicants strategy involves S1 and S2 sacral nerves, applicants also propose that both S1 and S2 cuffs (of the same side) be moulded together. Thus, the designed bi-cuff-electrode shown in FIG. 5 can host two nerves in distinct cuffs. Each cuff may have a different inner diameter and may contain up to 7 contacts.

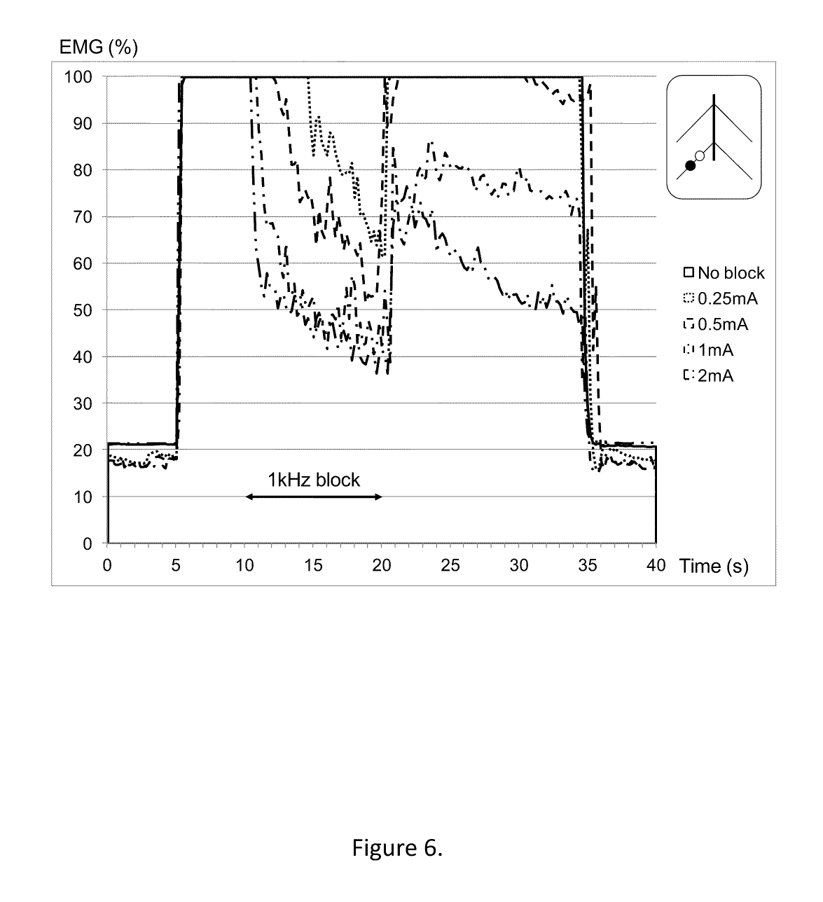

We present in this section results from 8 acute dog experiments carried out with the objective of verifying the potential benefit of the proposed strategy. The result of stimulation is observed with a real time recording of the intravesical and urethral pressures (PVES and PURA respectively) as well as the EUS and pelvic floor muscles EMG activity. FIG. 6 shows the EUS EMG activity during 30 sec of low-frequency stimulation of S2 nerve and 1 kHz distal blockade that is applied for 10 sec only after a delay of 5 sec. The EMG activity clearly decays when blockade is activated. If the blockade intensity is increased, the EMG decay is faster and reaches lower values. Once the blockade is stopped, the EMG returns back to a higher value as the low-frequency stimulation continues. However, using high amplitude currents seems to induce some fatigue as the EMG is lower and decreases after the blockade has been stopped. For higher blockade intensities, the EMG decay is faster and more important.

Procedure: Male mongrel dogs are subjected to laminectomy at the T10-11 level and spinal cord transection is done under general anesthesia and aseptic techniques, followed by a limited sacral laminectomy in order to expose the sacral roots. The ventral roots (S1-S3) of the sacral nerves supplying the bladder and the sphincter are separated extradurally and clearly identified by their anatomical arrangement as well as by electrical stimulation with hook electrodes while recording the intravesical and urethral pressure changes with computerized urodynamic equipment (Laborie Medical Technologies Inc—UDS-120). The urinary bladder is emptied and a 7-French triple lumen urethral catheter (C.R. Bard Inc.—Bard Urodynamic catheter) is inserted into the bladder. One channel of the catheter is used to monitor the intravesical pressure (PVES), the second channel to monitor the intraurethral pressure (PURA), and the third channel to fill the bladder when needed. The position of the catheter is confirmed by gently pressing on the bladder and the posterior urethra, which results in changes in PVES and PURA, respectively. The catheter is then secured to the foreskin to avoid displacement during the experiment. Electromyographic activity (EMG) of the external urethral sphincter and pelvic floor muscles are continuously recorded using needle electrodes and an EMG unit (Laborie Medical Technologies Inc—UDS-110). In general, S1 root is identified to be of a large diameter (1.5-2 mm) and gives rise to a marked increase in PURA with a minimal change in PVES. Stimulation of S2 nerve root gives mixed bladder and sphinteric responses, while stimulation of S3 nerve gives rise to almost no or a very weak change in both PVES and PURA. Then, according to the desired strategy, cuff-electrodes are wrapped around the targeted sacral roots. The bladder is slowly filled with saline to its full capacity then evacuated by neurostimulation. After the experiment, the dog under study is sacrificed by the animal care technician.

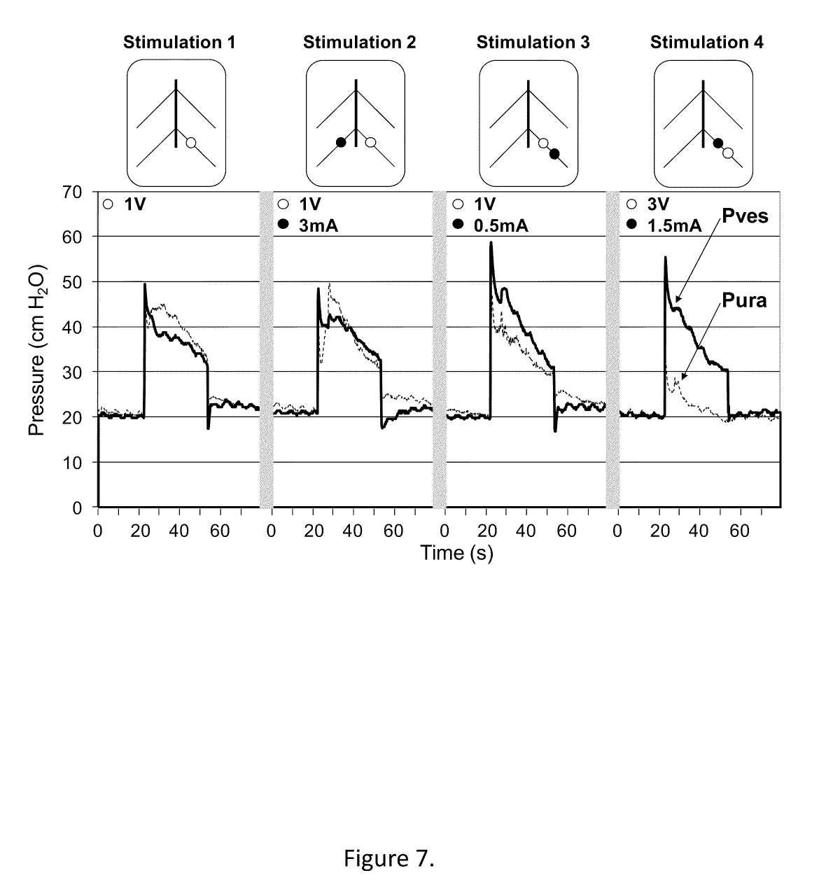

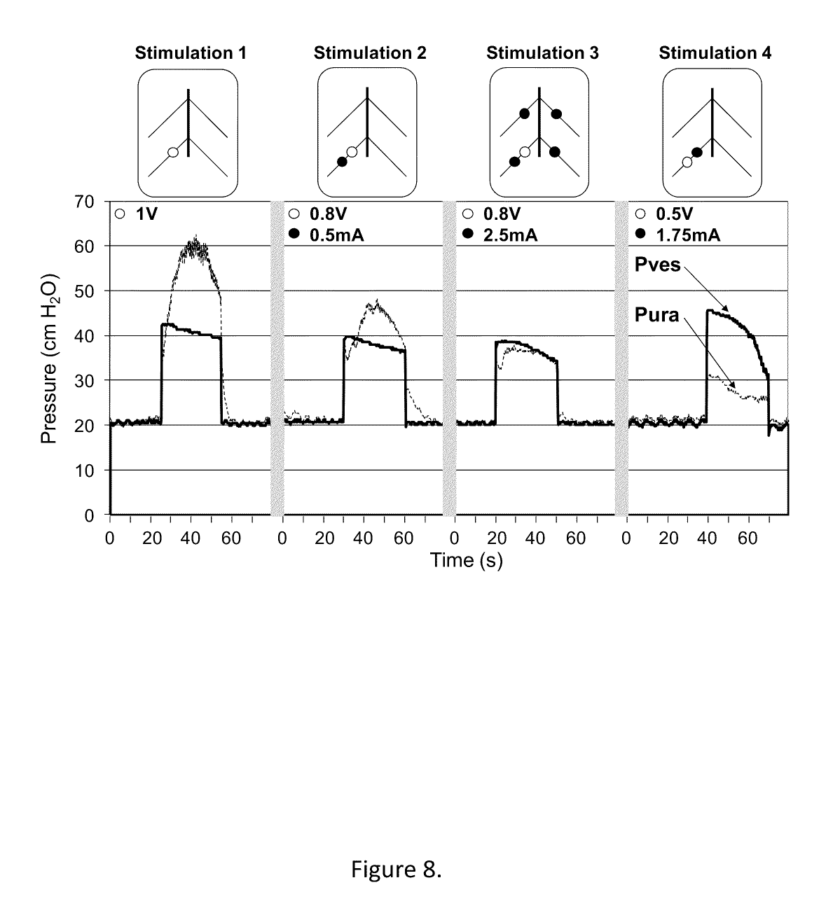

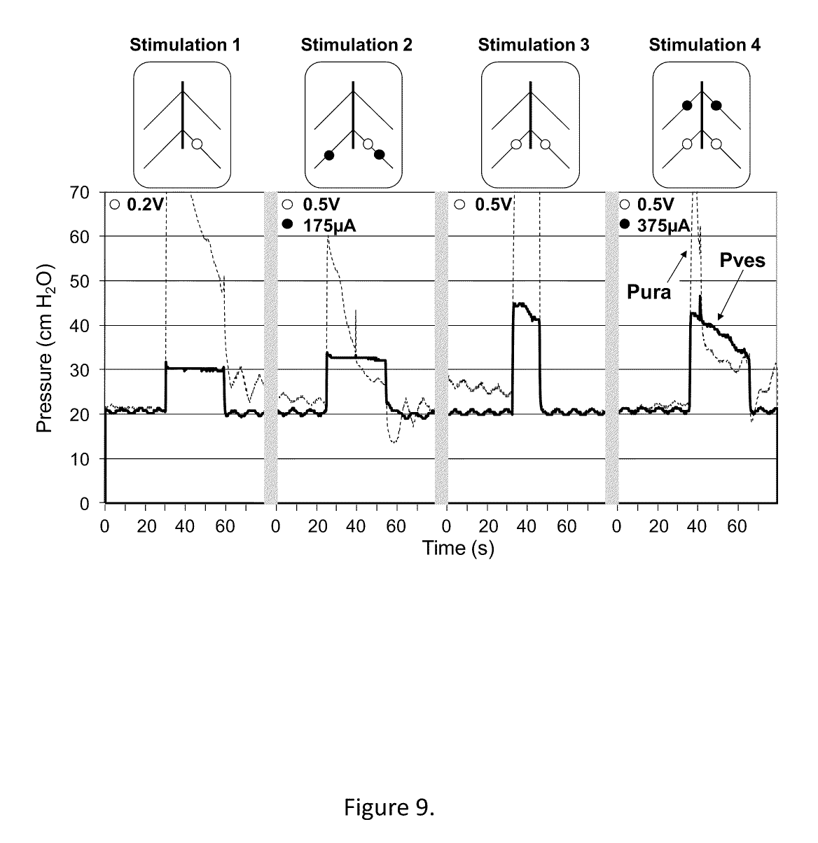

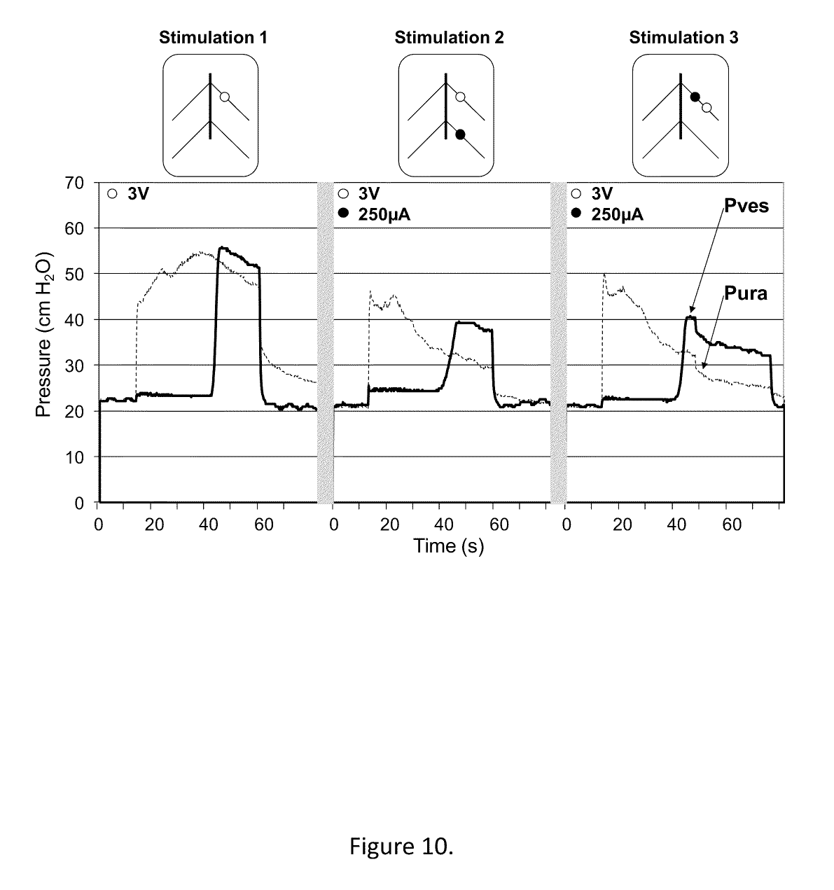

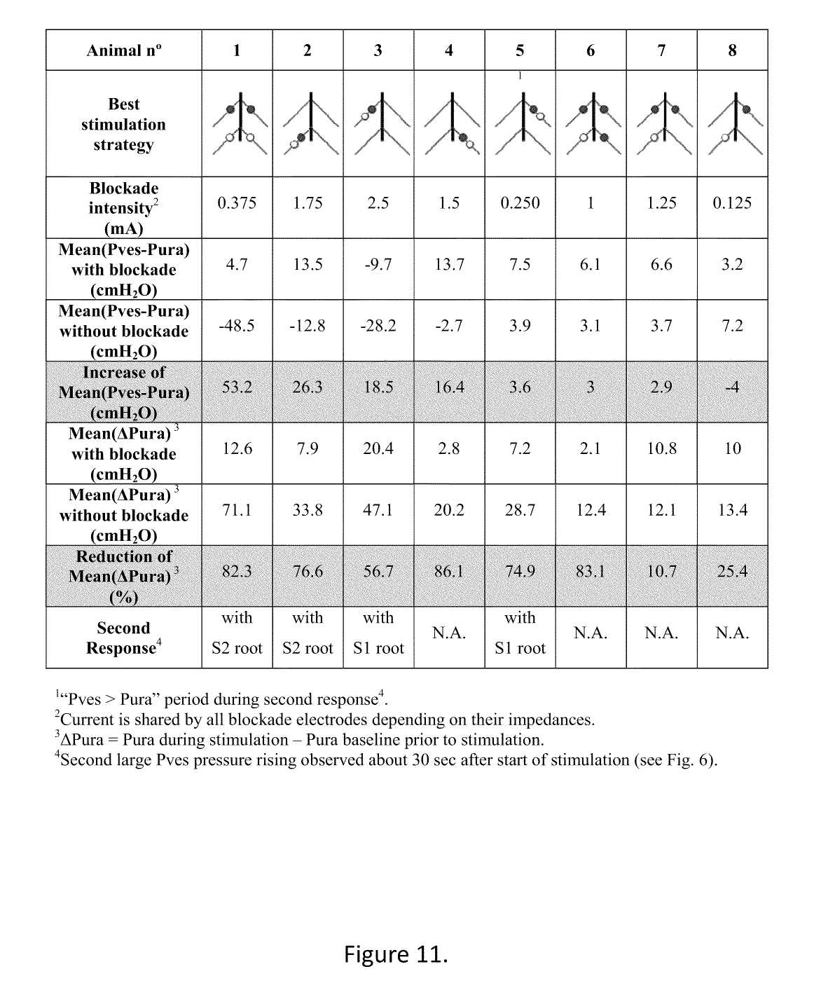

FIGS. 7-10 show stimulation sets from different animals: (FIG. 7) animal 4, (FIG. 8) animal 2, (FIG. 9) animal 1 and (FIG. 10) animal 5. “Stimulation 1” shows the response to conventional unilateral S2 low-frequency stimulation. “Stimulations 2 and 3” represent various configurations of stimulation/blockade. “Stimulation 4” is the best strategy that has been tested for that particular animal. They have been selected for being the most representative. In each experiment, applicants looked for the best stimulation strategy that would lead to an optimal micturition. That corresponds to a maximal rising of PVES associated with a maximal relaxation of the EUS which can be observed as a decrease of PURA. In FIG. 7, “Stimulation 1” shows the response to conventional unilateral S2 low-frequency stimulation, “Stimulations 2 and 3” represent various configurations of stimulation/blockade, whereas “Stimulation 4” gives the best strategy that has been tested.

In FIG. 7 (animal 4), the bladder response to unilateral stimulation (Right S2 nerve) was satisfactory with a maximum PVES increase of over 20 cmH2O (Stimulation 1). However, PURA remained higher than PVES preventing bladder emptying. Blockade of Left S2 nerve (Stimulation 2) led to a slight reduction of PURA. Distal blockade of Right S2 nerve (Stimulation 3) achieved a PVES higher than PURA, but both latter blockade types remained insufficient. It is a proximal blockade of the Right S2 nerve (Stimulations 4) that proved to be a very efficient strategy even without distal blockade. This means that, in this case, reflex EUS activation triggered by the low-frequency stimulation is dominant and should be blocked.

In FIG. 8 (animal 2), it is a unilateral stimulation of the Left S2 nerve that has been chosen to induce a satisfactory PVES response (Stimulation 1). In this case, PURA is much higher than PVES during stimulation. A distal blockade of Left S2 nerve (Stimulation 2) showed a significant decrease of PURA but still insufficient. Adding blockade on all other (S1 & S2) sacral nerves reduced even more PURA but it remained almost equal to PVES (Stimulation 3). Again, similarly to animal 4, it is a proximal blockade of the Left S2 nerve, without distal blockade, that proved to be efficient (Stimulations 4).

In FIG. 9 (animal 1), applicants observed a particularly high PURA peak that prevented us from increasing the Right S2 unilateral stimulation intensity to reach a satisfactory PVES amplitude (Stimulation 1). A simultaneous blockade of Left S2 nerve and a distal blockade of Right S2 nerve showed a significant decrease of PURA but PVES remained insufficient (Stimulation 2). Bilateral S2 stimulation was beneficial in increasing PVES (Stimulation 3) and the best strategy was to apply blockade on both S1 roots. This means that, in this case, reflex EUS activation involving S1 nerves (as opposite to the stimulated S2 nerves) is not only present but important to the point that it becomes necessary to block it.

In FIG. 10 (animal 5), “Stimulation 1” shows the response to Right S1 nerve low-frequency stimulation that is maintained for more than 45 sec. A large second PVES rising pressure occurs after a delay of about 30 sec, while no change has been made to the stimulation setup. Adding blockade as in “Stimulations 2 and 3” makes it an efficient strategy in this case. applicants had to stimulate the Right S1 nerve in order to obtain a response of the bladder (Stimulation 1). However, this response is quite different from that of common S2 stimulation. Interestingly, there is first, a small increase of PVES when low-frequency stimulation is switched on, then a second large PVES rising pressure occurs after a delay of about 30 sec, while no change has been made to the stimulation setup. This delayed rising pressure brings PVES to a value higher than PURA, and adding blockade as in Stimulations 2 and 3 makes it an efficient strategy in this case. Such a response was never reported in Applicants previous Polystim experiments as the duration of stimulation was limited to less than 15 sec in general. Applicants also observed this response, even though less important, in three other animals using S2 nerves in two of them. A longer stimulation of S1 or S2 nerves may trigger a spinal micturition reflex. A stimulus of 30 seconds in duration is considered to be a long stimulation duration. It will be appreciated by those skilled in the art that other durations may also be considered as long, even though the duration is more or less than 30 seconds.

A summary of best stimulation strategies and achieved results from the 8 acute dog experiments is presented in FIG. 11. For each animal, the best stimulation strategy is the one that led to a maximum (PVES-PURA) pressure difference that is maintained as long as possible during the target stimulation period of about 30 sec. Mean(PVES-PURA) and Mean(ΔPURA) values are both given with and without blockade. ΔPURA is PURA during stimulation minus PURA baseline prior to stimulation. The increase of Mean(PVES-PURA) and the percentage reduction of Mean(ΔPURA) give a measure of the achieved selectivity and EUS blockade success respectively. The animals are ordered with respect to the former. The achieved increase of Mean(PVES-PURA) ranged from −4 to 53.2 cmH2O, while the percentage reduction of Mean(ΔPURA) ranged from 10.7 to 86.1%. The blockade current intensity used in these cases ranged from 125 μA to 1.75 mA. When multiple blockade electrodes are used, blockade current is distributed depending on their impedances. Electrode impedances measured at 1 kHz ranged from 1.2 to 4.8 kΩ. In animal 8, even if blockade was observed, all utilized strategies did not improve the response. Among the best stimulation strategies, three of them involved bilateral blockade of S1 nerves (animals 1, 6, 7), and four others involved proximal blockade of the same stimulated nerve (animals 2-5)

Applicants have demonstrated a new sacral multisite stimulation strategy based on nerve conduction blockade using high-frequency stimulation as an alternative to rhizotomy. This approach aims at increasing bladder contraction selectively and decreasing DSD. Thus, better micturition could be achieved while preserving sexual and defecation reflexes as well as sacral sensations, if still present in case of incomplete SCI. Acute dog experiments were carried out to test the proposed strategy and EUS blockade has been achieved in 8 animals. Given the eventually high number of electrodes required for this strategy, a custom multiple-contacts bi-cuff-electrode design has also been developed to be applied around two nerves in distinct cuffs. Following these experiments, the main observations are that high-frequency blockade can be very efficient in reducing the EUS resistance and that the optimal strategy is different from one animal to another. Results show an interesting potential benefit of the proposed strategy in decreasing DSD without any rhizotomy.

It will be appreciated that due to the inappropriate experimental setup resulting from a faulty device, data from dog #9 was not considered in the results and should not influence the conclusions from the other 8 dogs which had proper experimental setup. Dogs have two important sacral nerves for the neurostimulation of the urinary system. In dogs, it has been observed that stimulation of S1 and/or S2 induces a response of the bladder and/or sphinter muscles. It is understood that applicants have performed experimental work on animals, such as dogs, but that applicants anticipate the data to be extrapolated to humans. It will be appreciated by those skilled in the art that stimulation of different sacral nerves could be required to achieve adequate micturition reflex in humans. If more sacral nerves are involved in the neurostimulation of the urinary system in humans, a multiple-cuff electrode could be designed following the same approach as the bi-cuff electrode. The objective of implanting only one electrode is to minimize invasiveness of the operation and to limit the number of independent devices implanted.

It will be appreciated by those skilled in the art that, due to inter-subject variation, determining an optimal stimulation strategy can require performing test stimulations in order to observe their effect on bladder micturition function, where bladder function can be evaluated by the intravesical-intraurethral pressure difference, total volume of evacuated urine, or any other method known in the art. Test stimulations having high and low frequency stimulations as well as stimulations in different chronological order and at different locations on a nerve may be required to determine an optimal stimulation strategy.

What is claimed is:

1. A method to enhance micturition in paraplegics by sacral neurostimulation involving a combination of a low-frequency electrical stimulation applied to one or more sacral nerves to induce bladder contraction and a high frequency electrical stimulation applied to at least one sacral nerve to cause nerve conduction blockade that prevents urethral sphincter dyssynergic contraction.

2. The method of claim 1, wherein the high-frequency signal at least partially disrupts the afferent sensory signals.

3. The method of claim 1, wherein the high-frequency signal at least partially disrupts the efferent large diameter fibres innervating the urethral sphincter.

4. The method of claim 1, wherein the high-frequency signal is delivered to the sacral nerve at a location found between the bladder and the low-frequency stimulation site.

5. The method of claim 1, wherein the high-frequency signal is delivered to the sacral nerve at a location found between the spinal-cord and the low-frequency stimulation site.

6. The method of claim 1, wherein said electrical stimulation is delivered from an implant device via at least one electrode attached to a patient.

7. The method of claim 1, wherein said electrical stimulation is performed at any one of said sacral nerves.

8. The method of claim 1, wherein said electrical stimulation is bilateral.

9. The method of claim 1, wherein the high frequency stimulation is performed at a frequency greater than 300 Hz.

10. The method of claim 1, wherein the high frequency stimulation is performed near 1 kHz.

11. The method of claim 1, wherein the low frequency stimulation is performed at a frequency smaller than 300 Hz.

12. The method of claim 1, wherein the low frequency stimulation is at about 30 Hz.

13. The method of claim 1 wherein the current amplitude is between 5 μA and 5 mA

14. The method of claim 1, wherein one electrode applies a low-frequency signal to the one or more ventral sacral root and another electrode applies a high-frequency signal to one or more dorsal sacral root.

15. The method of claim 1, wherein stimulation is performed in the extradural space.

16. The method of claim 1, further comprising performing test stimulations to identify an optimal stimulation strategy.

17. The method of claim 1, wherein stimulation of S1 or S2 nerves is prolonged to a long duration, and a spinal micturition reflex is increased at long duration over short duration.

18. A neurostimulator for enhancing bladder function having at least two electrical signal sources and electrodes for coupling to sacral nerves, the neurostimulator being configured to deliver a combination of a low-frequency electrical stimulation to one or more sacral nerves to induce bladder contraction and a high frequency electrical stimulation to at least one sacral nerve to cause nerve conduction blockade that prevents urethral sphincter dyssynergic contraction.

19. A neurostimulator cuff for securing around at least two nerves comprising:
an electrically non-conductive main body having a central portion for receiving electrical input, said main body having at least two projections defining at least two nerve channels for receiving a nerve, and wherein said nerve channels can be in an open or closed position;
an electrically conductive material comprising one or more electrode engaging said central portion of said main body and distributed independently along each nerve channel to make electrical contact along said at least two nerves.

20. The neurostimulator cuff of claim 19 wherein the electrically conductive material is stainless steel.

21. The neurostimulator cuff of claim 19, wherein said cuff is biased in the closed position.

22. The neurostimulator cuff of claim 19, wherein said cuff is biased in the open position and further comprises a closure clip for ensuring proper closure around nerve.

23. The neurostimulator cuff of claim 19 wherein the non-conducting portion is vinyl polysiloxane material.
